(19) United States
(12) Patent Application Publication
(10) Pub. No.: US 2014/0366893 A1
(43) Pub. Date: Dec. 18, 2014

(54) FOOD OBSTRUCTING DENTAL DEVICE

(71) Applicants: Elaine Marques, Newark, NJ (US); Miquel Marques, Newark, NJ (US)

(72) Inventors: Elaine Marques, Newark, NJ (US); Miquel Marques, Newark, NJ (US)

(21) Appl. No.: 14/303,743

(22) Filed: Jun. 13, 2014

Related U.S. Application Data
(60) Provisional application No. 61/835,030, filed on Jun. 14, 2013.

(51) Int. Cl. A61F 5/00

(52) U.S. Cl. CPC A61F 5/0006
USPC 128/861

(57) ABSTRACT

A food obstructing dental device comprising a palate cover, where the palate cover is positioned toward a palate within a user's mouth, and where the palate cover includes an edge shaped to follow an arch behind the user's teeth; and a pair of teeth attachments attached to a lateral portion of the palate cover, where the pair of teeth attachments fasten around a pair of molars with the user's mouth to secure the palate cover within the mouth, wherein when the palate cover is attached with the pair of teeth attachments a portion of the volume of the user's mouth is obstructed.

### CROSS REFERENCE TO OTHER APPLICATIONS

This application claims priority to U.S. Provisional Application Ser. No. 61/835,030 filed on Jun. 14, 2013.

### BACKGROUND OF THE INVENTION

1. Field of the Invention

The present invention relates to a retainer like mouthpiece positioned on the roof of the mouth with attachments points around the molar teeth to reduce the volume within the mouth.

2. Description of Related Art

Many people are continuously looking to lose weight whether it's the last 10 pounds or a significant amount over 30 pounds. Some people may be trying to lose weight to look better in their clothes or to lift their self-esteem. Alternatively, others are losing weight for health reason whether doctor advised or to improve their overall health. In general, when people are at their ideal weight their bodies can run most efficiently not having to combat the problems associated with excess body fat.

To assist people with weight loss, diet plans, weight loss pills, and specialized exercise equipment all advertise helping people to lose weight. While these programs may help a person in the short term, the optimal way to lose weight is with a caloric deficit; by simply consuming less calories than are used in a day. While the basic rule is simple, actually putting the reduced calorie advice into practice is difficult.

Therefore, it would be beneficial in the art to provide a device that assists a person to reduce the calories they intake. It would also be desirable in the art to provide a device that is easy to use for convenience to the user.

### SUMMARY OF THE INVENTION

In view of the foregoing disadvantages inherent in the prior art, the general purpose of the present invention is to provide a food obstructing dental device to assist a user with weight loss, configured to include all of the advantages of the prior art, and to overcome the drawbacks inherent therein.

Accordingly, an object of the present invention is to provide a food obstructing device with a palate cover which obstructs a user's mouth cavity thereby slowing their food consumption.

Another object of the present invention is to provide a food obstructing device that attaches to the back molars within the user's mouth for a secure fit within the mouth.

To achieve the above objects, in an aspect of the present invention, a food obstructing dental device is described a comprising a palate cover, where the palate cover is positioned toward a palate within a user's mouth, and where the palate cover includes an edge shaped to follow an arch behind the user's teeth; and a pair of teeth attachments attached to a lateral portion of the palate cover, where the pair of teeth attachments fasten around a pair of molars with the user's mouth to secure the palate cover within the mouth, wherein when the palate cover is attached with the pair of teeth attachments a portion of the volume of the user's mouth is obstructed.

These together with other aspects of the present invention, along with the various features of novelty that characterize the present invention, are pointed out with particularity in the claims annexed hereto and form a part of this present invention. For a better understanding of the present invention, its operating advantages, and the specific objects attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description and claims taken in conjunction with the accompanying drawing in which:

FIG. 1 depicts a perspective view of a food obstructing dental device in accordance with an exemplary embodiment of the present invention.

### DETAILED DESCRIPTION OF THE DRAWINGS

The present invention relates to a dental device positioned on the roof of the mouth with attachments points around the molar teeth to reduce the volume within the mouth. The present invention provides a food obstructing dental device with tooth attachments to make a user feel full faster by reducing the amount of food consumed in a sitting. The food obstructing dental device reduces the food intake by forcing the user to chew at a slower rate, therefore feeling full faster. The food obstructing dental device includes an acrylic palate cover with two bands that attach around two molars in the mouth. During use, the user simply inserts the dental device and attaches it to the molars. The dental device causes the user to chew slower and take smaller bites which is ideal for users trying to lose weight.

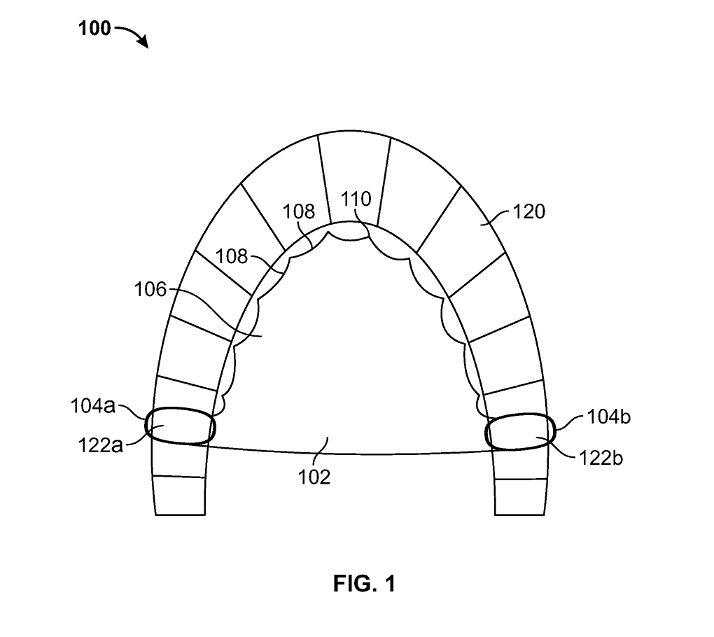

Turning now descriptively to the drawing, referring to FIG. 1, a perspective view of a food obstructing dental device 100 is shown in accordance with an exemplary embodiment of the present invention. The food obstructing dental device 100 includes a palate cover 102 that obstructs access to the palate or roof of the user's mouth. The palate cover 102 comprises a generally semicircular shape to follow the arch of the user's mouth from the front incisors to the back molars. The palate cover 102 may be made from an acrylic, plastic, rubber, polyurethane or similar material able to endure the repeated insertion and chewing exerted on the dental device 100.

The palate cover 102 includes an edge 106 that is shaped as an arch to follow behind the user's teeth 120. The edge 106 may be smooth with rounded edges to ensure a comfortable feel in the user's mouth. Alternatively, the edge 106 may include a plurality of depressions 108 to create a scalloped edge around the palate cover 102. The depressions 108 receive each individual tooth 120 within the mouth for a custom fit. A plurality of points 110 are positioned between each depression 108. The points 110 may insert slightly between the user's teeth 120 to ensure that the food obstructing mouthpiece 100 does not move or shift during use. The palate cover 102 may be made from a mold of the user's upper teeth and palate for a fit that is exact to their individual mouth.

Attached to the lateral edges of the palate cover 102 is a pair of teeth attachments 104a, 104b. The teeth attachments 104a, 104b fasten around the molars 122a, 122b within the user's mouth. The molars 122a, 122b provide a strong and stable base for the food obstructing dental device 100 to ensure that the palate cover 102 does not move during use. The teeth attachments 104a, 104b may be metal bands or wires like those used in braces and other common dental appliances. For use the user inserts the food obstructing dental device 100 into their mouth and fits teeth attachments 104a, 104b around the molars 122a, 122b. The palate cover 102 is pressed towards the roof of the mouth until a comfortable fit is obtained with the mouth. With the food obstructing dental device 100 the user is afforded an easy to use weight loss device that assists the user to consume less by simply obstructing the amount of food they are able to eat at a time. By blocking a portion of the volume of the mouth, the user takes smaller bits, chews slower, feels full faster and in turn consumes less resulting losing weight.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A food obstructing dental device comprising:
a. a palate cover, where the palate cover is positioned toward a palate within a user's mouth, and where the palate cover includes an edge shaped to follow an arch behind the user's teeth; and
b. a pair of teeth attachments attached to a lateral portion of the palate cover, where the pair of teeth attachments fasten around a pair of molars with the user's mouth to secure the palate cover within the mouth,
wherein when the palate cover is attached with the pair of teeth attachments a portion of the volume of the user's mouth is obstructed.

2. The food obstructing dental device according to claim 1, where the edge of the palate cover is scalloped.

3. The food obstructing dental device according to claim 2, where the scalloped edge includes a plurality of depressions and a plurality of points, where the plurality of depressions receive the user's teeth, and where the plurality of points are directed between the user's teeth.

4. The food obstructing dental device according to claim 1, where the pair of teeth attachments are bands.

5. The food obstructing dental device according to claim 1, where the pair of teeth attachments are wires.

6. The food obstructing dental device according to claim 1, where the palate cover is made from one of at least acrylic, plastic, rubber or polyurethane.

7. The food obstructing dental device according to claim 1, where the palate cover is made from a mold of the user's upper teeth and palate for a custom fit.
